(19) United States
(12) Patent Application Publication
(10) Pub. No.: US 2011/0112637 A1
(43) Pub. Date: May 12, 2011

(54) INTRAOCULAR LENS

(75) Inventors: Lászlo Kontur, Munich (DE); Nándor Turkevi-Nagy, Zsambek (HU)

(73) Assignee: Medicontur Orvostechnikai Korlatolt Felelossegu Ta rsasag, Zsambek (HU)

(21) Appl. No.: 12/993,487

(22) PCT Filed: May 19, 2009
(86) PCT No.: PCT/EP2009/056084

§ 371 Date: Jan. 25, 2011

(30) Foreign Application Priority Data
May 21, 2008 (FR) 0802761

(51) Int. Cl. A61F 2/16

(52) U.S. Cl. 623/6.43

(57) ABSTRACT

Intraocular lens (1, 101) with an optic part (2) and a haptic part (3, 103), characterized in that it includes, at each transition between the optic part (2) and the haptic part (3, 103), two notches (7a, 8a, 14, 15, 122, 123), the two notches (7a, 8a, 14, 15, 122, 123) being arranged respectively on either side of the transition, each notch (7a, 8a, 14, 15, 122, 123) extending along the edge of the haptic part (3, 103) and impinging on the periphery of the optic part (2).

The subject of the present invention is an intraocular lens.

An intraocular lens comprises an optic part, forming the actual corrective optical system, and a haptic part, which is used for the fitting and fixing of the optic part inside the eye in the appropriate position.

Such a lens is generally produced in a flexible material so that it can be bent or rolled, to be able to be inserted into the eye via a small incision, the lens returning to its normal shape and dimensions when it is fitted in the eye.

Intraocular lenses have to address a certain number of mechanical constraints. In particular, the haptic part of the lens must be flexible enough to strain the capsular sac correctly regardless of the size of the eye in which the lens is fitted. The haptic part of the lens must, however, be sufficiently rigid in the optical plane to allow for effective correction.

The shape of the intraocular lens must be suitable to limit the migration of cells, the latter leading to a posterior capsule opacification (PCO). To counter the posterior capsule opacification, a square edge is generally produced over the entire periphery of the optic part.

For this reason, intraocular lenses are generally produced by milling and then turning. The milling is used to cut the lens from a block of flexible material and to produce the square edge on the periphery of the optic part. Then, the turning is used to produce the square edge at the transitions between the optic part and the haptic part. This may lead to off-centering problems when changing tools.

The aim of the present invention is to propose an intraocular lens which addresses these mechanical constraints, which avoids the off-centering problems during fabrication and which limits the migration of cells.

To this end, the subject of the invention is an intraocular lens comprising an optic part and a haptic part, characterized in that it comprises, at each transition between said optic part and said haptic part, two notches, said two notches being arranged respectively on either side of said transition, each notch extending along the edge of said haptic part and impinging on the periphery of said optic part.

According to a first embodiment of the invention, said haptic part comprises two haptic assemblies arranged symmetrically relative to the center of said optic part, each of said two haptic assemblies including two loops, each of said two loops comprising a void portion surrounded by a rim.

Preferably, in the first embodiment of the invention, each of said void portions is included in a triangle oriented so that the two axes surrounding the apex of the triangle situated alongside said optic part intersect a diameter of said optic part, above the optical axis of said optic part.

Advantageously, in the first embodiment of the invention, each of said void portions impinges on the periphery of said optic part.

Preferably, in the first embodiment of the invention, said set of notches includes four notches, each notch having a rounded shape, extending along the edge of one of said loops, and impinging on the periphery of said optic part.

According to a second embodiment of the invention, said haptic part comprises two diametrically opposite elements, each of said elements including a contact portion, intended to come into contact with the internal wall of the eye, and a connecting arm, connecting said element to the periphery of said optic part.

Preferably, in the second embodiment of the invention, said set of notches includes at least two notches, each notch having an ovoid shape, extending along an edge of one of said connecting arms, and impinging on the periphery of said optic part.

The invention will be better understood, and other aims, details, features and advantages thereof will become more clearly apparent from the following detailed explanatory description of several embodiments of the invention, given as purely illustrative and non-limiting examples, with reference to the appended schematic drawings.

In these drawings:

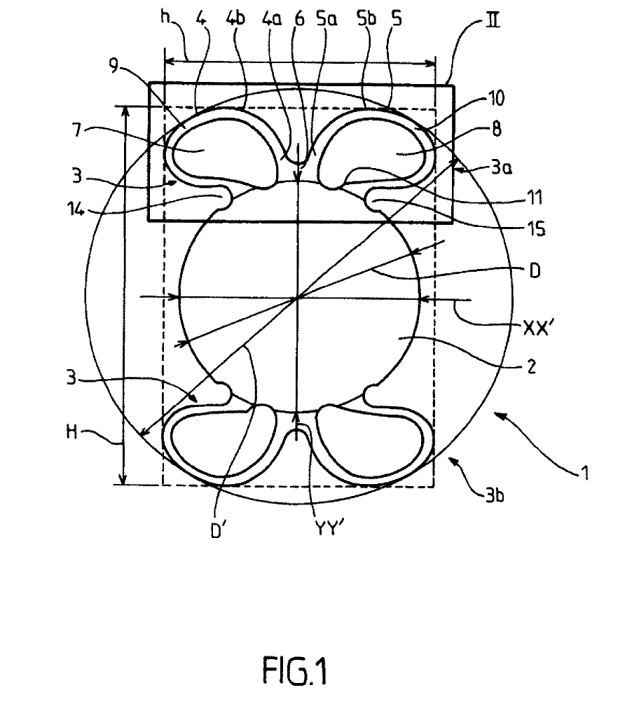

FIG. 1 is a simplified schematic view in elevation of the posterior face of an intraocular lens according to a first embodiment of the invention;

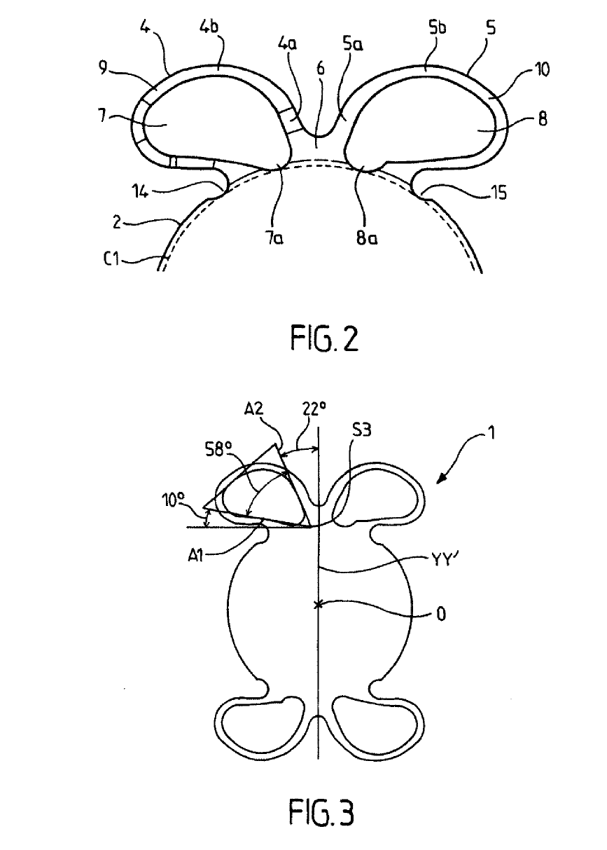

FIG. 2 is an enlarged view of the region II of FIG. 1;

FIG. 3 is a view similar to FIG. 1 specifying the shape of a loop of the haptic part of the intraocular lens;

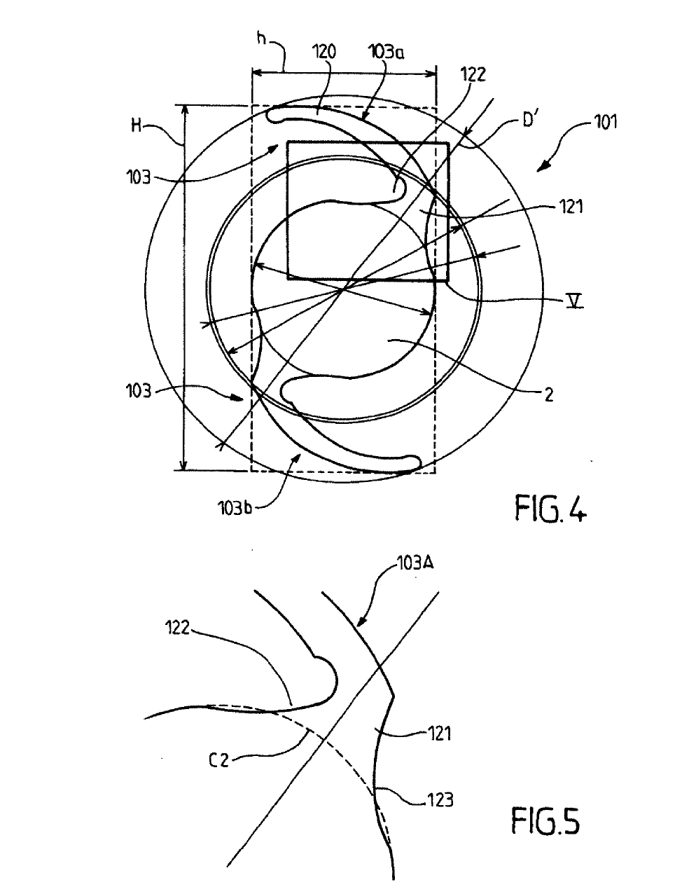

FIG. 4 is a view similar to FIG. 1 showing an intraocular lens according to a second embodiment of the invention; and

FIG. 5 is an enlarged view of the region V of FIG. 4.

FIG. 1 shows a single-piece flexible intraocular lens 1 according to a first embodiment of the invention. The lens 1 is intended to be implanted in the capsular sac of an eye (not represented), in a manner that is known per se.

The intraocular lens 1 has an optic part 2 and a haptic part 3. For example, the intraocular lens 1 has a height H of approximately 9.74 mm, a width h of approximately 6.68 mm, and can be included in a circle of diameter D′ approximately equal to 10.7 mm. Generally, the dimensions of the intraocular lens 1 are suited to the dimensions of a capsular sac.

The optic part 2 is substantially circular and its diameter D is, for example, approximately equal to 6 mm.

The haptic part 3 comprises two haptic assemblies 3a and 3b arranged symmetrically relative to the center of the optic part 2; that is to say, relative to the intersection of a diameter X, X′ of the optic part 2 and of a diameter Y, Y′ of the optic part 2. The two haptic assemblies 3a, 3b are identical, so only the haptic assembly 3a is described in detail hereinbelow.

The haptic assembly 3a comprises two loops 4 and 5, each loop 4, 5 having a first end 4a, 5a, respectively, connected to the periphery of the optic part 2, and a second end 4b, 5b, respectively, elastically deformable, intended to come into contact with the internal wall of the eye when the lens 1 is fitted in the eye.

The two loops 4, 5 are arranged substantially symmetrically relative to the diameter Y, Y′. The haptic assembly 3a includes a linking strip 6 extending between the two loops 4 and 5 along the periphery of the optic part 2.

Each loop 4, 5 comprises a void portion 7, 8, respectively, which is surrounded by an elastically deformable rim 9, 10, respectively. Each rim 9, 10 typically has a width of approximately 0.22 mm. One of the void portions 7, 8, for example the void portion 8, includes a polarizer 11.

Referring to FIG. 3, it can be seen that each void portion 7, 8 is included in a triangle oriented so that the two axes A1 and A2 surrounding the apex S3 of the triangle situated alongside the optic part 2 intersect the diameter Y-Y′ above the optical axis O. This shape and this orientation of the void portions 7, 8 have the effect of reducing the compression forces which are exerted on the loops 4, 5 when the lens 1 is fitted in the eye. It has been found from experimentation that the compression forces are reduced by approximately a factor of two relative to the forces exerted on the intraocular lenses of the prior art. Furthermore, this shape and this orientation of the loops 4, 5 prevents, or at least limits, the presence of folds in the capsular sac, which avoids the migration of cells and therefore the appearance of PCO (posterior capsule opacification).

Each void portion 7, 8 forms a notch 7a, 8a, respectively, which impinges on the periphery of the optic part 2, for example over a thickness of approximately 0.1 mm. This has the effect of reducing the risk of the passage of cells, and therefore the appearance of PCO. FIG. 2 shows by broken lines an arc of circle C1 of the same center as the optic part 2 to show the shrinkage of material on the periphery of the optic part 2, that is to say the notches 7a, 8a.

The lens 1 includes, at the level of each loop 4, 5, a notch 14, 15, respectively, of rounded shape, which extends along the edge of the loop 4, 5 and impinges on the periphery of the optic part 2, for example over a thickness of approximately 0.1 mm (FIG. 2). The notches 14, 15 have three functions.

In other words, the lens 1 comprises, at each transition between the optic part 2 and the haptic part 3, two notches 7a, 8a, 14, 15. The two notches 7a, 8a, 14, 15 are arranged respectively on either side of the transition, each notch 7a, 8a, 14, 15 extending along the edge of the haptic part 3 and impinging on the periphery of the optic part 2.

Firstly, the notches 14, 15 serve as centering means during the production of the lens 1, which avoids the off-centering problems when changing tools, and therefore makes it possible to reduce the number of unusable lenses. In practice, the notches 7a, 8a, 14, 15 are used to separate the working line of the tools, that is to say the turning and milling tools, into a number of segments. Each segment can thus be calibrated separately, which facilitates centering.

Secondly, the presence of notches 14, 15 causes material to be eliminated, which has the effect of limiting the migration of cells, and therefore the appearance of PCO. In practice, it has been observed from experimentation that the cells have a tendency to migrate along the periphery of the optic part 2, and then pass over the lens 1 at a transition between the optic part 2 and the haptic part 3. The notches 7a, 8a, 14, 15 make it possible to definitely have a square edge at this transition, and thus form a reservoir retaining the cells and preventing their migration over the lens 1.

Thirdly, the notches 14, 15 create an inflexion point enabling the loops 4, 5 to be moved away toward the exterior at the time of fitting of the lens 1, which makes it possible to enhance the securing of the lens 1 in the capsular sac.

FIG. 4 shows an intraocular lens 101 according to a second embodiment. The elements that are similar to the first embodiment are given the same reference numeral and will not be described again.

Here, the intraocular lens 101 has, for example, a height H of approximately 12.30 mm, a width h of approximately 6 mm, and can be included in a circle of diameter D′ approximately equal to 13 mm.

The haptic part 103 comprises two diametrically opposite elements 103a and 103b. Since the two elements 103a and 103b are identical, only the element 103a will be described hereinbelow. The element 103a includes a contact portion 120, intended to come into contact with the internal wall of the eye, and a connecting arm 121 connecting the element 103a to the periphery of the optic part 2.

The lens 101 includes a notch 122, of ovoid shape, which extends along an edge of the arm 121 and impinges on the periphery of the optic part 2. The lens 101 includes a notch 123, of ovoid shape, which extends along the other edge of the arm 121 and impinges on the periphery of the optic part 2.

The lens 101 therefore includes, at each transition between the optic part 2 and the haptic part 103, two notches 122, 123. The two notches 122, 123 are arranged respectively on either side of the transition, each notch 122, 123 extending along the edge of the haptic part 103 and impinging on the periphery of the optic part 2.

The notches 122 and 123 have the same functions as the notches 14, 15 described previously. FIG. 5 shows, by broken lines, an arc of circle of the same center and of the same radius as the optic part 2 to show the shrinkages of material on the periphery of the optic part 2.

Although the invention has been described in relation to a number of particular embodiments, it is obvious that it is by no means limited and that it includes all the technical equivalents of the means described and their combinations if the latter fall within the context of the invention.

1. An intraocular lens (1, 101) comprising an optic part (2) and a haptic part (3, 103), characterized in that it comprises, at each transition between said optic part (2) and said haptic part (3, 103), two notches (7a, 8a, 14, 15, 122, 123), said two notches (7a, 8a, 14, 15, 122, 123) being arranged respectively on either side of said transition, each notch (7a, 8a, 14, 15, 122, 123) extending along the edge of said haptic part (3, 103) and impinging on the periphery of said optic part (2).

2. The intraocular lens as claimed in claim 1, characterized in that said haptic part (3) comprises two haptic assemblies (3a, 3b) arranged symmetrically relative to the center of said optic part (2), each of said two haptic assemblies including two loops (4, 5), each of said two loops comprising a void portion (7, 8) surrounded by a rim (9, 10).

3. The intraocular lens as claimed in claim 2, characterized in that each of said void portions (7, 8) is included in a triangle oriented so that the two axes (A1, A2) surrounding the apex (S3) of the triangle situated alongside said optic part (2) intersect a diameter (YY′) of said optic part, above the optical axis (0) of said optic part.

4. The intraocular lens as claimed in claim 2, characterized in that each of said void portions (7, 8) impinges on the periphery of said optic part (2).

5. The intraocular lens as claimed in claim 2, characterized in that said set of notches includes four notches (14, 15), each notch having a rounded shape, extending along the edge of one of said loops (4, 5), and impinging on the periphery of said optic part (2).

6. The intraocular lens as claimed in claim 1, characterized in that said haptic part (103) comprises two diametrically opposite elements (103a, 103b), each of said elements including a contact portion (120), intended to come into contact with the internal wall of the eye, and a connecting arm (121), connecting said element to the periphery of said optic part (2).

7. The intraocular lens as claimed in claim 6, characterized in that said set of notches includes at least two notches (122, 123), each notch having an ovoid shape, extending along an edge of one of said connecting arms (121), and impinging on the periphery of said optic part (2).

8. The intraocular lens as claimed in claim 3, characterized in that each of said void portions (7, 8) impinges on the periphery of said optic part (2).

9. The intraocular lens as claimed in claim 3, characterized in that said set of notches includes four notches (14, 15), each notch having a rounded shape, extending along the edge of one of said loops (4, 5), and impinging on the periphery of said optic part (2).

10. The intraocular lens as claimed in claim 4, characterized in that said set of notches includes four notches (14, 15), each notch having a rounded shape, extending along the edge of one of said loops (4, 5), and impinging on the periphery of said optic part (2).

11. The intraocular lens as claimed in claim 8, characterized in that said set of notches includes four notches (14, 15), each notch having a rounded shape, extending along the edge of one of said loops (4, 5), and impinging on the periphery of said optic part (2).
